(19) United States
(12) Patent Application Publication
(10) Pub. No.: US 2014/0012128 A1
(43) Pub. Date: Jan. 9, 2014

(54) PATIENT SUPPORT APPARATUS HAVING A TRANSFER PLATE AND A MAGNETIC RESONANCE DEVICE HAVING THE PATIENT SUPPORT APPARATUS

(71) Applicant: Patrick Gross, Buckenhof (DE)

(72) Inventor: Patrick Gross, Buckenhof (DE)

(21) Appl. No.: 13/921,280

(22) Filed: Aug. 1, 2013

(30) Foreign Application Priority Data
Jul. 4, 2012 (DE) 102012211658.3

(51) Int. Cl. A61B 5/055

(52) U.S. Cl. CPC A61B 5/0555
USPC 600/415

(57) ABSTRACT

A patient support apparatus includes a base unit, a reclining couch relative to the base unit, a transfer plate which can be fixed on the reclining couch and a signal transmission unit for data transmission and/or a signal transmission purposes between the transfer plate and the reclining couch. The signal transmission unit includes an inductive coupling unit.

### CROSS REFERENCE TO RELATED APPLICATIONS

The present application relates to German Patent Office application No. 102012211658.3 DE filed Jul. 4, 2012, the entire content of which is hereby incorporated herein by reference.

### FIELD OF INVENTION

The present invention relates to a patient support apparatus, for in particular a magnetic resonance device, having a base unit, a reclining couch relative to the base unit, a transfer plate which can be fixed on the reclining couch and a signal transmission unit for data transmission and/or signal transmission purposes between the transfer plate and the reclining couch.

### BACKGROUND OF INVENTION

With surgical interventions, such as for instance neurosurgical interventions, a catheter intervention etc., it is known to perform a magnetic resonance examination on the patient immediately after the surgical intervention and/or during an interruption in the surgical intervention. To this end, the patient is arranged and/or positioned on a transfer plate and, together with this transfer plate, is moved from an operating table to a magnetic resonance device. Transportation of the patient together with the transfer plate is herewith effected by means of a patient support apparatus, which has a reclining couch for receiving the transfer plate.

Local coils are usually attached to the patient for the magnetic resonance examination. These local coils must be connected to the patient support apparatus and/or the magnetic resonance device for the magnetic resonance examination and are unplugged again after the magnetic resonance examination.

For such interventions, it is necessary, on account of patient safety, to perform the magnetic resonance examination with a minimal expenditure of time. On account of the time pressure and/or an emergency situation, this may nevertheless result in particular in clinical operating personnel forgetting to unplug connections between the local coils. This may result in an uncontrolled pulling of cables and/or in a tearing of the cables and in this way in mechanical risk to the patient and/or damage to the local coils and/or the plug-in contacts.

### SUMMARY OF INVENTION

The object underlying the present invention is in particular to provide a patient support apparatus, by means of which a workflow for a magnetic resonance examination of a patient positioned on a transfer plate is optimized. The object is achieved by means of the features of the independent claim. Advantageous embodiments are described in the dependent claims.

The invention relates to a patient support apparatus, for in particular a magnetic resonance device, having a base unit, a reclining couch relative to the base unit, a transfer plate which can be fixed on the reclining couch and a signal transmission unit for data transmission and/or signal transmission purposes between the transfer plate and the reclining couch.

It is proposed that the signal transmission unit includes an inductive coupling unit, as a result of which a cable-free and/or wireless data transmission and/or signal transmission can advantageously be achieved between the transfer plate and the reclining couch. Furthermore, with magnetic resonance examinations, a plugging-in of accessory units, in particular of plugs and/or connections of local coils, prior to the magnetic resonance examination, and an unplugging of these accessory units can advantageously be omitted. In such a way, a workflow of the operating personnel can be optimized for the magnetic resonance examination in such a way and an examination period can be advantageously reduced. In this context, a transfer plate is to be understood to mean in particular a plate for mounting and/or positioning the patient, wherein the transfer plate can be exchanged between different patient support apparatuses and/or operating tables. By way of example, the patient reclining on the transfer plate can firstly be arranged on an operating table for a surgical intervention and then together with the transfer plate on a patient support apparatus for a subsequent magnetic resonance examination. For a magnetic resonance examination, the patient, reclining on the transfer plate, is introduced, together with the transfer plate and the reclining couch, into an examination room of the magnetic resonance device, wherein to this end the transfer plate is arranged in a position which is stopped opposite to the reclining couch. Furthermore, an inductive coupling unit is in particular to be understood to mean a coupling unit, in which the signals and/or an energy are transmitted in a contactless manner, in particular without galvanic coupling, by means preferably of an electromagnetic induction. On account of the contactless transmission of signals, it is advantageously also possible to dispense with a grounding of the transfer plate and/or the accessory units, in particular the local coils, which further contributes to a compact and space-saving patient support apparatus.

Furthermore, it is proposed that the inductive coupling unit comprises at least one transformer unit, as a result of which a particularly cost-effective and/or space-saving inductive coupling unit can be realized for an inductive coupling of the transfer plate with the reclining couch. To this end the transformer unit particularly preferably comprises at least one first winding unit, which is arranged on the transfer plate and a second winding unit, which is arranged on the reclining couch.

A particularly space-saving arrangement of the winding unit on the transfer plate with a minimal transmission path between the two winding units and/or between the transfer plate and the reclining couch can be achieved if the winding unit is arranged on a side of the transfer plate facing the reclining couch. Furthermore, an undesirable restriction in a reclining surface of the transfer plate which is available to the patient can herewith advantageously be prevented.

In an advantageous development of the invention, it is proposed for the transfer plate to be stopped on the reclining couch in an examination position, wherein the winding unit arranged on the reclining couch and the winding unit arranged on the transfer plate have the shortest distance from one another in the examination position of the transfer plate. A consistently high transmission quality of the signals and/or data to be transmitted can herewith be achieved. The shortest distance can in this way be embodied all the larger, the better the two winding units are attuned to one another. Furthermore, with a consistent distance between the two winding units, a greater amplification of the signal to be transmitted can be achieved, the better the two winding units are attuned to one another. In this context, a shortest distance is to be understood in particular as a distance between the winding unit arranged on the transfer plate and the winding unit arranged on the reclining couch, wherein the distance along a direction of movement of the transfer plate with a movement of the transfer plate in respect of the reclining couch assumes a minimal value.

It is further proposed that the signal transmission unit comprises at least one signal amplification unit, which is arranged on the transfer plate. An advantageous signal-to-noise ratio can herewith be achieved and thus interferences and noise signals can be suppressed. The signal amplification unit is herewith preferably arranged along a signal transmission path from the transfer plate to the reclining couch in front of the winding unit arranged on the transfer plate.

For a flexible and mobile supply of power to a power supply of the inductive coupling unit and/or the amplifier unit, provision can be made for the signal transmission unit to comprise at least one energy storage means. Furthermore, it is advantageously possible to dispense with a power supply line for the signal transmission unit and a particularly compact signal transmission unit is made available. The energy storage means is preferably formed by a conventional, in particular rechargeable battery, so that a particularly cost-effective signal transmission unit can also be made available for the patient support apparatus.

Furthermore, it is proposed that the amplifier unit be configured so as to change a frequency of an incoming signal, as a result of which undesirable feedback during the signal transmission from the accessory unit, in particular the local coil, to the transfer plate, can particularly advantageously be prevented. To this end, the amplifier unit preferably comprises a frequency modulation unit and/or further adjusting elements for changing and/or adjusting the frequency of the incoming signal. An incoming signal is to be understood here in particular to mean a signal which is sent for instance by an accessory unit, in particular a local coil, to the winding unit arranged on the transfer plate.

In a further embodiment of the invention, it is proposed that the transfer plate be embodied translucently in respect of a high frequency radiation, as a result of which the transfer plate is advantageously embodied in a magnetic resonance compatible manner. The transfer plate is preferably also embodied to be particularly thin with a thickness of less than 5 cm, preferably less than 3 cm and particularly advantageously less than 2 cm, so that the transfer plate has a high transmission property in respect of the high frequency radiation and/or of high frequency signals. On account of this embodiment of the invention, the transformer unit, in particular the winding unit of the transformer unit arranged on the transfer plate, can also be arranged on a side of the transfer plate facing away from the reclining couch and nevertheless an advantageous inductive coupling can be achieved between the two winding units of the transfer plate and of the reclining couch.

The invention is further based on a magnetic resonance device having a patient support apparatus as claimed in one of claims 1 to 10. A workflow of the operating personnel can be optimized for the magnetic resonance examination in such a way and an examination period can be advantageously reduced. Furthermore, with magnetic resonance examinations, a plugging-in of accessory units, in particular of plugs and/or connections of local coils, prior to the magnetic resonance examination, and an unplugging of these accessory units can advantageously be omitted. Furthermore, a cable-free coupling for data transmission and/or signal transmission purposes can advantageously be achieved between the transfer plate and the reclining couch.

### BRIEF DESCRIPTION OF THE DRAWINGS

Further advantages, features and details of the invention will emerge from the exemplary embodiment described below and with the aid of the drawing.

In the figures:

FIG. 1 shows an inventive patient support apparatus in a schematic representation and

FIG. 2 shows a schematic representation of a magnetic resonance device with the patient support apparatus.

### DETAILED DESCRIPTION OF INVENTION

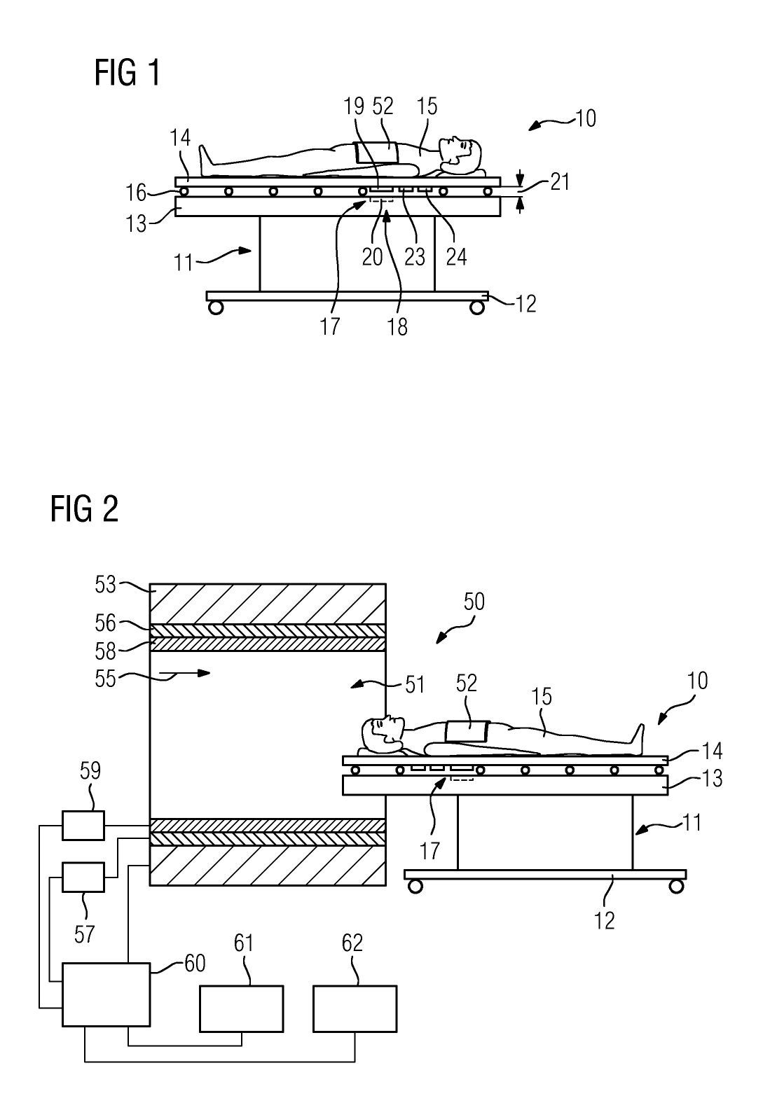

FIG. 1 shows a schematic representation of a patient support apparatus 10 according to the invention. The patient support apparatus 10 includes a base unit 11 with a running gear unit 12, so that the patient support apparatus 10 can be embodied to be movable. Furthermore, the patient support apparatus 10 includes a reclining couch 13 and a transfer plate 14. The reclining couch 13 is embodied to be movable in respect of the base unit 11, in order for instance to move a patient 15 into an examination area 51 of a magnetic resonance device 50.

The transfer plate 14 is embodied to be movable in respect of the reclining couch 13 and is arranged on the reclining couch 13. In an examination position of the transfer plate 14, the transfer plate 14 is stopped on the reclining couch 13, wherein to this end the reclining couch 13 and/or the transfer plate 14 comprise stop elements (not shown in more detail). In this stopped position in respect of the reclining couch 13, the transfer plate 14 can only be moved in respect of the base unit together with the reclining couch 13. A movement of the reclining couch 13 together with the transfer plate 14 stopped in the examination position takes place for instance for a magnetic resonance examination, in which the patient 15 reclining on the transfer plate 14 is positioned within the examination region 51.

For a movement of the transfer plate 14 in respect of the reclining couch 13, the patient support apparatus 10 comprises sliding and/or bearing elements 16, such as for instance roller elements, which are arranged between the transfer plate 14 and the reclining couch 13. The patient 15 can be interchanged between different patient support apparatuses 10 and/or operating tables using the transfer plate 14. The transfer plate 14 with the patient 15 reclining and/or positioned on the transfer plate 14 is herewith interchanged between the different patient support apparatuses 10 and/or the operating tables.

Furthermore, the patient support apparatus 10 comprises a signal transmission unit 17 for data transmission and/or signal transmission between the transfer plate 14 and the reclining couch 13. The signal transmission unit 17 includes an inductive coupling unit 18 for cable-free and/or wireless data transmission and/or signal transmission purposes In this way, signals and/or data can be transmitted from an accessory unit 53, such as for instance a local coil, via the transfer plate 14 onto the reclining couch 13, without to this end a galvanic connection being required between the accessory unit 53 and/or the transfer plate 14 and the reclining couch 13. The reclining couch 13 is thus embodied galvanically separated from the accessory unit 53 and/or the transfer plate 14.

The inductive coupling unit 18 comprises a transformer unit for the cable-free and/or wireless coupling between the transfer plate 14 and the reclining couch 13. The transformer unit comprises a first winding unit 19 and a second winding unit 20. The first winding unit 19 of the transformer unit is arranged on the transfer plate 14. The first winding unit 19 is arranged on a side of the transfer plate 14 facing the reclining couch 13 in a particularly space-saving manner. Alternatively to this, the first winding unit 19 is also arranged on a side of the transfer plate 14 facing away from the reclining couch 13.

The second winding unit 20 of the transformer unit is arranged on the reclining couch 13, wherein the second winding unit 20 is herewith preferably arranged on a region of the reclining couch 13 facing the transfer plate 14.

In the present exemplary embodiment, the embodiment of the inductive coupling unit 18 is restricted to a single transformer unit. In alternative embodiments of the invention, inductive coupling units 18 with two or more transformer units are however also conceivable, without herewith exceeding the scope of protection of the invention.

The transfer plate 14 is embodied to be translucent in respect of a high frequency radiation, so that undesirable interferences during a magnetic resonance examination are herewith prevented. In addition, the transfer plate 14 has a thickness 21 of less than 5 cm, preferably less than 3 cm and particularly advantageously less than 2 cm, so that a high radiation permeability of high frequency radiation is provided.

In an examination position of the transfer plate 14 on the reclining couch 13, the transfer plate 14 is stopped with the reclining plate 13, so that the transfer plate 14 can only still be moved together with the reclining couch 13. In this examination position, the first winding unit 19 arranged on the transfer plate 14 and the second winding unit 20 arranged on the reclining couch 13 have a consistently short distance from one another. This shortest distance of the two winding units 19, 20 amounts to approximately 1 cm to a few cm. The shortest distance between the two winding units 19, 20 is nevertheless less than 8 cm, preferably less than 5 cm and particularly advantageously less than 2 cm.

On account of the arrangement of the first winding unit 19 on the transfer plate 14 and the second winding unit 20 on the reclining couch 13, the distance between the two winding units 19, 20 increases with a movement of the transfer plate 14 in respect of the reclining couch 13.

The signal transmission unit 17 further comprises a signal amplification unit 23 and an energy storage means 24. The energy storage means 24 is preferably formed of conventional, in particular rechargeable batteries. The amplifier unit 23 is arranged along a signal transmission direction and/or a data transmission direction from the first winding unit 19 to the second winding unit 20 upstream of the first winding unit 19 of the transformer unit. Incoming signals, in particular signals emitted from the accessory unit 53, for instance from a local coil, are amplified by means of the amplifier unit 23 prior to transmission to the transformer unit.

Furthermore, the amplifier unit 23 is configured such that a frequency of an incoming signal can be changed by means of the amplifier unit 23. The frequency of the incoming signal is herewith modulated prior to transmission by means of the transformer unit so that undesirable feedbacks during the signal transmission are prevented. To this end, the amplifier unit 23 can comprise a modulation unit and/or further adjusting elements for adjusting the frequency.

A subarea, in particular the subarea comprising the first winding unit 19, the amplifier unit 23 and the energy storage means 24, the signal transmission unit 17, is arranged on the transfer plate 14 and a further subarea, in particular the subarea including the second winding unit 20, the signal transmission unit 17 is arranged on the reclining couch 13. The subarea of the signal transmission unit 17 arranged on the transfer plate 14 is arranged along a transverse extension of the transfer plate 14 essentially in a central region of the transfer plate 14, wherein the transverse extension is aligned at right angles to a direction of movement of the transfer plate 14 in respect of the reclining couch 13.

Furthermore, the reclining couch 13 also comprises a depression, along its transverse extension, which is aligned at a stopped position of the transfer plate 14 in parallel to the transverse extension of the transfer plate 14, in a central region, said depression extending along the longitudinal extension of the reclining couch 13, in order to receive the sub area of the signal transmission unit 17 arranged on the transfer plate 14 with a movement of the transfer plate 14 in respect of the reclining couch 13.

The subarea of the signal transmission unit 17 arranged on the reclining couch 13 is preferably also arranged along the transverse extension of the reclining couch 13 in the central region.

FIG. 2 illustrates the magnetic resonance device 50 in more detail. The magnetic resonance device 50 includes a magnet unit 53 having a main magnet 54 for generating a strong and in particular constant main magnetic field 55. Furthermore, the magnetic resonance device 50 has the cylinder-shaped examination area 51 for receiving a patient 15 for the magnetic resonance examination, wherein the examination area 51 is enclosed in a circumferential direction by the magnet unit 53. The patient 15 can be moved into the examination area 51 by means of the patient support apparatus 10.

The magnet unit 53 additionally has a gradient coil 56 for generating magnetic field gradients which is used for spatial encoding during an imaging session. The gradient coil 56 is controlled by means of a gradient control unit 57. Furthermore, the magnet unit 53 comprises a high frequency antenna unit 58 and a high frequency antenna control unit 59 for exciting a polarization which develops in the main magnetic field 55 generated by the main magnet 54. The high frequency antenna unit 58 is controlled by the high frequency antenna control unit 59 and radiates high frequency magnetic resonance sequences into an examination area 51.

In order to control the main magnet 54, the gradient control unit 57 and to control the high frequency antenna control unit 59, the magnetic resonance device 50 comprises a control unit 60 formed of a computing unit. The computing unit centrally controls the magnetic resonance device 50, such as for instance the implementation of a predetermined imaging gradient echo sequence. Control information such as for instance imaging parameters as well as reconstructed magnetic resonance images can be indicated to an operator on a display unit 61, for instance on at least one monitor, of the magnetic resonance device 50. Furthermore, the magnetic resonance device 50 comprises an input unit 62, by means of which information and/or parameters can be input by an operator during a measuring process.

1. A patient support apparatus, comprising:
a base unit,
a reclining couch relative to the base unit,
a transfer plate which can be fixed on the reclining couch and a signal transmission unit for data transmission and/or a signal transmission purposes between the transfer plate and the reclining couch,
whererin the signal transmission unit includes an inductive coupling unit.

2. The patient support apparatus as claimed in claim 1,
wherein the inductive coupling unit comprises at least one transformer unit.

3. The patient support apparatus as claimed in claim 2, wherein the transformer unit comprises at least one winding unit, which is arranged on the transfer plate.

4. The patient support apparatus as claimed in claim 3, wherein the winding unit is arranged on a side of the transfer plate facing the reclining couch.

5. The patient support apparatus as claimed in claim 2, wherein the transformer unit comprises at least one winding unit, which is arranged on the reclining plate.

6. The patient support apparatus as claimed in claim 3, wherein the transfer plate is stopped on the reclining couch in an examination position, wherein the winding unit arranged on the reclining couch and the winding unit arranged on the transfer plate have a shortest distance from one another in the examination position of the transfer plate.

7. The patient support apparatus as claimed in claim 5, wherein the transfer plate is stopped on the reclining couch in an examination position, wherein the winding unit arranged on the reclining couch and the winding unit arranged on the transfer plate have a shortest distance from one another in the examination position of the transfer plate.

8. The patient support apparatus as claimed in claim 1, wherein the signal transmission unit comprises at least one signal amplification unit, which is arranged on the transfer plate.

9. The patient support apparatus as claimed in claim 1, wherein the signal transmission unit comprises at least one energy storage means.

10. The patient support apparatus as claimed in claim 9, wherein the amplifier unit is configured to change a frequency of an incoming signal.

11. The patient support apparatus as claimed in claim 1, wherein the transfer plate is embodied to be translucent in respect of a high frequency radiation.

12. The patient support apparatus as claimed in claim 1, wherein the patient support apparatus is part of a magnetic resonance device.

13. A magnetic resonance device, comprising:
a patient support apparatus as claimed in claim 1.
